(19) United States
(12) Patent Application Publication
(10) Pub. No.: US 2013/0158382 A1
(43) Pub. Date: Jun. 20, 2013

(54) Medical Treatment System With Non-Coplanar Capability

(71) Applicant: David Chao, New York, NY (US)

(72) Inventor: David Chao, New York, NY (US)

(21) Appl. No.: 13/715,876

(22) Filed: Dec. 14, 2012

Related U.S. Application Data
(60) Provisional application No. 61/576,327, filed on Dec. 15, 2011.

(51) Int. Cl. A61N 5/10

(52) U.S. Cl. CPC A61N 5/1081
USPC 600/407; 600/1

(57) ABSTRACT

Systems and methods for delivering coplanar and non-coplanar medical treatments are described in which a rotating member is engaged with an angularly displaced treatment ring including at least one treatment device, whereby rotation of the rotating member and the treatment ring or treatment device combine to allow non-coplanar therapy to be delivered to a patient.

### RELATED APPLICATION

This application claims the benefit of U.S. Provisional Application No. 61/576,327 filed Dec. 15, 2011, titled “Non-Parallel Double Ringed Apparatus For Non-Coplanar Focused Therapy,” which is hereby incorporated by reference.

### TECHNICAL FIELD

The present application relates to systems and methods for medical treatment and particularly to the delivery of noninvasive medical treatments from a system capable of co-planar and non-coplanar delivery.

### BACKGROUND

The disclosed medical therapy system provides an elegant and efficient design for both coplanar and non-coplanar therapy. In the example where the system is utilized to deliver radiation treatment, its non-coplanar beams provide for improved treatment with lower doses to surrounding healthy tissues, due to the increased number of angles from which beams can be directed. The system's configuration is similarly beneficial for high-frequency focused ultrasound therapy, focused radio frequency therapy, laser therapy, ablation and other noninvasive therapies.

### SUMMARY

Medical treatment systems and methods are disclosed, some embodiments of which may include a stand, a rotating member engaged with the stand and configured to rotate in a first rotation plane, a treatment ring configured to rotate in a second rotation plane, which is engaged with and angularly displaced from the rotating member by a tilt angle, where the first rotation plane and the second rotation plane are angularly separated by the tilt angle, the at least one treatment device engaged with the treatment ring and configured to deliver therapy to a patient and wherein rotation of the rotating member causes the at least one treatment device to follow a non-coplanar path for delivering therapy.

Other embodiments may include a stand, a rotating member engaged with the stand and configured to rotate in a first rotation plane, a treatment ring engaged with and angularly displaced from the rotating member by a tilt angle and having a first axis, at least one treatment device to deliver therapy to a patient engaged with the treatment ring and configured to rotate around the first axis in a second rotation plane, wherein the first rotation plane and the second rotation plane are angularly separated by the tilt angle and wherein rotation of the rotating member causes the at least one treatment device to follow a non-coplanar path for delivering therapy to the patient.

In some embodiments, the at least one treatment device may be configured to deliver a therapy such as radiation therapy, high-frequency focused ultrasound therapy, ablative therapy, laser therapy or radio-frequency therapy.

In other embodiments the tilt angle may be variable or fixed.

In further embodiments, the system further includes a beam stopper engaged with the treatment ring and configured to rotate in the second rotation plane, opposite the at least one treatment device.

In still further embodiments, the at least one treatment device is configured to rotate and deliver therapy to targets displaced from the isocenter of the treatment ring.

In some embodiments, the system may further include a diagnostic quality imaging device and a couch configured to move the patient from the isocenter to the diagnostic quality imaging device.

In other embodiments, the system may also include at least one imaging device engaged with the treatment ring and located opposite the at least one treatment device.

In other embodiments, the system may further include at least one imaging device engaged with the treatment ring and located adjacent the at least one treatment device.

In another embodiment, the system may also include a protective wall around the patient.

These and other features, aspects, and advantages of the present disclosure will become better understood with reference to the following description and claims.

### BRIEF DESCRIPTION OF THE DRAWINGS

Features, aspects, and embodiments of the disclosure are described in conjunction with the attached drawings, in which:

FIG. 1A shows a medical treatment system with a treatment head located 180 degrees from a pivot point.

FIG. 1B shows the medical treatment system of FIG. 1A with the treatment head rotated 180 degrees from its position in FIG. 1A.

FIG. 2A shows the medical treatment system of FIG. 1A with the treatment head located 0 degrees from a pivot point.

FIG. 2B shows the medical treatment system of FIG. 2A with the treatment head rotated 180 degrees from its position in FIG. 2A

FIG. 3 shows examples of a coplanar and two non-coplanar beam paths that may be generated by the system of this disclosure.

FIG. 4 is an alternative embodiment and shows additional detail of an exemplary medical treatment system similar to the system of FIG. 1.

FIG. 5 shows the medical treatment system of FIG. 1 with an optional protective wall around the patient.

### DETAILED DESCRIPTION

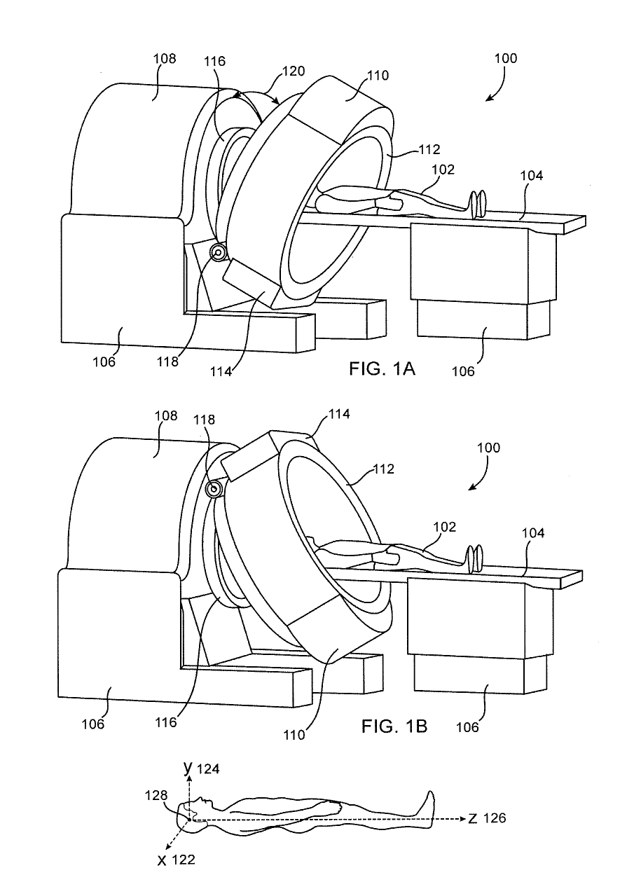

In FIG. 1A, the medical treatment system 100 for treating a patient 102 includes a movable patient couch 104 on which patient 102 lies. The entire system is supported by a stand 106 and includes a housing 108. The treatment system 100 and stand 106 are preferably constructed in a manner that does not require any indentation in the floor of the room or treatment vault in which it is placed. Medical treatments are delivered from treatment head 110 located on treatment ring 112. While the term ring is used to describe treatment ring 112, cross sections other than a perfect circle may be used in accordance with the spirit of this disclosure. Depending upon the particular medical treatment being administered, the treatment head 110 may include a treatment device such as a radiation therapy beam source, for example, an x-ray, gamma ray, electron, proton, heavy-ion or any other type of beam generator. Examples of preferred embodiments include within treatment head 110, a linear accelerator or a radioisotope beam source. Alternatively, treatment head 110 may include a high-frequency focused ultrasound device or other devices for noninvasive therapy, etc.

It is contemplated that treatment ring 112 may include multiple treatment heads 110 in order to, for example, further increase the speed of treatment delivery or compensate for a lower power treatment device. The multiple treatment heads 110 may take any geometric arrangement around treatment ring 112 and may include any type of treatment device. One potential embodiment involving multiple treatment heads 110 comprises four equally spaced heads including radioisotope beam sources for radiation therapy. Another embodiment involves multiple treatment heads 110 including high-frequency focused ultrasound devices for ablative therapy.

Opposite treatment head 110 on treatment ring 112 may be located a beam stopper 114 used to intercept a radiation beam from treatment head 110, as may be used in certain embodiments. Beam stopper 114 is used to limit ambient radiation concerns to secondary scatter from patient 102 and couch 104 and to leakage from the radiation source. Beam stopper 114 may be made of metal materials including, but not limited to, lead and tungsten and their alloys. Preferably, the thickness of beam stopper 114 is chosen to reduce radiation by a factor of 1000, so that leakage radiation at one meter does not exceed 0.1% of the primary beam at isocenter 128. In the case where there are multiple treatment heads 110, the preferred embodiment includes multiple beam stoppers 114 across treatment ring 112 from the multiple heads.

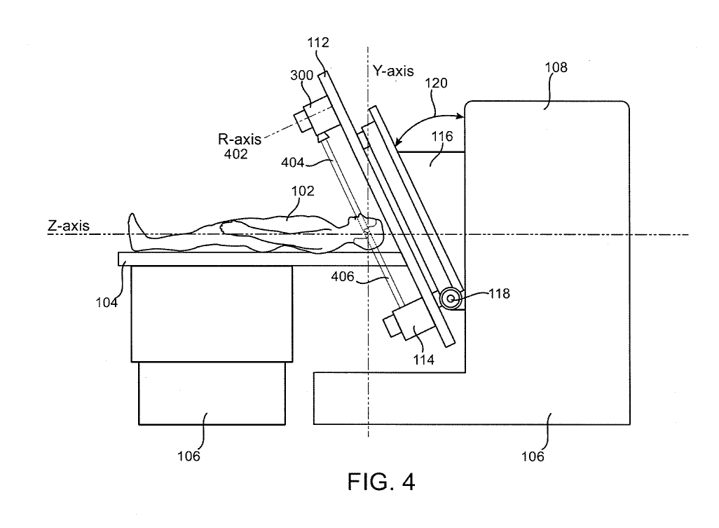

Treatment ring 112 is connected to a rotating member 116 and angularly displaced from rotating member 116 by a tilt angle 120 so that treatment ring 112 and rotating member 116 may rotate in non-parallel planes. In one embodiment, the connection between treatment ring 112 and rotating member 116 includes a pivot point 118. The pivot point may be a pin, hinge or any other mechanism facilitating the tilting of treatment ring 112 in relation to rotating member 116, although, in the alternative embodiment shown in FIG. 4, pivot point 118 may be fixed at a given tilt angle 120, and can merely be the description of the intersection of the rotating member 116 and the treatment ring 112. The connection between rotating member 116 and treatment ring 112 may be a direct connection, or the two may be connected through another member, such as a pivot point. Rotating member 116 may be a cylinder centered on the Z-axis as depicted in FIG. 1 or may be any other structure capable of rotating pivot point 118 through a 360 degree path.

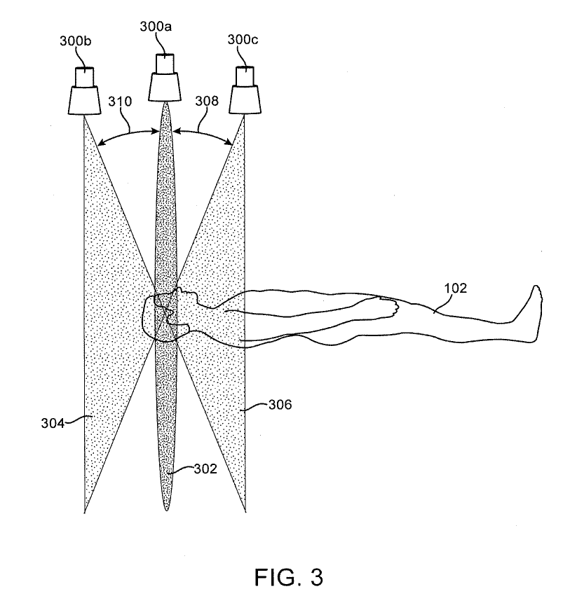

FIG. 1A shows treatment head 110 positioned at a location 180 degrees away from pivot point 118. FIG. 1B then illustrates the system after a 180 degree rotation of rotating member 116, as may occur during the delivery of a treatment. When treatment head 110 is in this position with respect to pivot point 118, a conical non-coplanar path 306, shown in FIG. 3, represents all of the possible treatment angles achievable during a rotation of rotating member 116. When treatment head 110 is in this position, cone angle 308 will be equal to tilt angle 120.

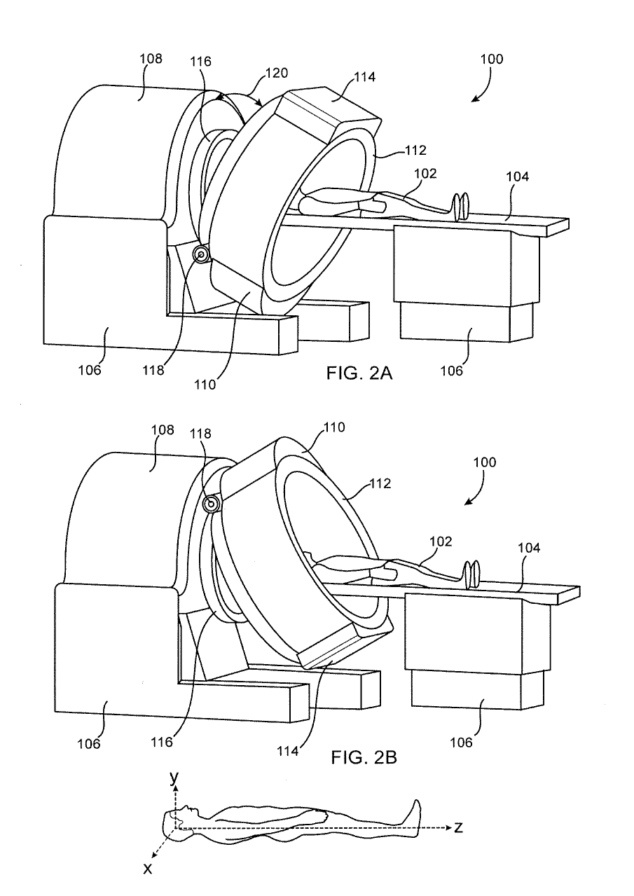

FIG. 2A shows treatment head 110 positioned at a location 0 degrees away from pivot point 118, after a 180 degree rotation of treatment ring 112 from its position shown in FIG. 1A. FIG. 2B then illustrates the system after a 180 degree rotation of rotating member 116, as may occur during the delivery of a treatment. When treatment head 110 is in this position with respect to pivot point 118, a conical non-coplanar path 304, shown in FIG. 3, represents all of the possible treatment angles achievable during a rotation of rotating member 116. When treatment head 110 is in this position, cone angle 310 will be equal to tilt angle 120.

When treatment head 110 is positioned at a location 90 degrees away from pivot point 118, the system will be capable of delivering coplanar treatments. Coplanar path 302 depicted in FIG. 3 represents all of the possible treatment angles achievable during a rotation of the rotating member 116 in the coplanar position.

When treatment head 110 is positioned at any location other than 0, 90 or 180 degrees from pivot point 118, non-coplanar treatments are possible along conical paths as depicted in FIGS. 3 at 304 and 306, but with cone angles 308 or 310 less than tilt angle 120. If tilt angle 120 is increased, the maximum cone angle 308/310 likewise increases to allow non-coplanar treatment at greater angles.

System 100 may be designed such that tilt angle 120 between rotating member 116 and treatment ring 112 can be varied, via a pivoting motion at pivot point 118. In such an embodiment, in addition to the connection at pivot point 118, there may be an additional translating support member opposite pivot point 118, or in multiple locations around treatment ring 112, that help support the weight of treatment ring 112. System 100 will have a maximum tilt angle 120 determined by the diameter of treatment ring 112 and the space needed for clearance of patient 102. Hard-stop or other safety mechanisms may be employed to ensure that tilt angle 120 does not increase to the point where treatment ring 112 could collide with patient 102.

System 100 may also be designed such that treatment ring 112 is fixed at a particular tilt angle 120 with respect to rotating member 116 as depicted in FIG. 4. In the embodiment of FIG. 4, rotating member 116 is fixed to treatment ring 112 at pivot point 118 and also across the back surface of treatment ring 112 so that tilt angle 120 cannot be varied. In one preferred embodiment, tilt angle 120 is fixed at 35 degrees. Even when tilt angle 120 is fixed, non-coplanar treatments at different cone angles 308/310 can be administered by rotating treatment ring 112 to position treatment head 110 at different angular displacements from pivot point 118 (or by rotating treatment head 300 in the case where treatment device 300 can rotate independently of treatment ring 112).

One or more treatment devices 300 are associated with the one or more treatment heads 110 and treatment ring 112. In one embodiment, treatment devices 300 are fixed to a particular circumferential location on treatment ring 112 and thus rotate along with treatment ring 112. In another embodiment, treatment ring 112 does not rotate about its own axis, but instead, treatment devices 300 rotate about the axis of treatment ring 112, within treatment ring 112. This rotation may be accomplished, for example, by treatment devices 300 moving along circumferential tracks within treatment ring 112.

Treatment device or devices 300 are typically arranged to direct their therapy at isocenter 128 at the center of treatment ring 112. However, when a therapy target is not located along the Z-axis or the centerline of patient 102, treatment device 300 may be rotated around the R-axis 402 depicted in FIG. 4 in order to treat such a target. The system 100 may thus be configured to be able to treat multiple targets, whether they are located at isocenter 128, or displaced from isocenter 128, without having to move the patient. This capability may also minimize or eliminate the need to move patient couch 104 to treat such targets that are displaced from isocenter 128. In this embodiment, beam stopper 114 can be made larger to cover a greater portion of the circumference of treatment ring 112 in order to ensure the absorption of the beam remnants when the beam is directed to a location off of isocenter 128.

Treatment device 300 may include a collimation device such as a multi-leaf collimator, radiosurgery cone or the like to focus treatment beam 404, and may also include a beam stopper 114 to absorb attenuated beam 406 that may pass completely through patient 102. System 100 may further include an imaging device across from treatment device 300, capable of providing imaging based on attenuated beam 406 passing through patient 102. In the case where treatment device 300 provides a photon treatment beam, the imaging device may be a megavoltage x-ray imaging panel. The imaging device may be placed in front of beam stopper 114 or in place of it. Alternatively, system 100 may include a kilovoltage source or sources placed on treatment ring 112 with kilovoltage x-ray imaging panels on the opposite side of treatment ring 112. Such panels may allow for computed tomography through movement of treatment head 110 by rotation of either treatment ring 112 or of rotating member 116. The imaging device or devices placed in treatment ring 112 may provide real-time patient location information during the treatment process.

System 100 may also include a diagnostic quality imaging device, such as a CT, MRI, PET, SPECT, and the like. This device may be located within housing 108 and, in a preferred embodiment, is a diagnostic quality CT scanner. Patient couch 104 may be translatable in the direction of the Z axis a sufficient distance to move patient 102 within the diagnostic quality imaging device to allow for improved patient location and treatment planning

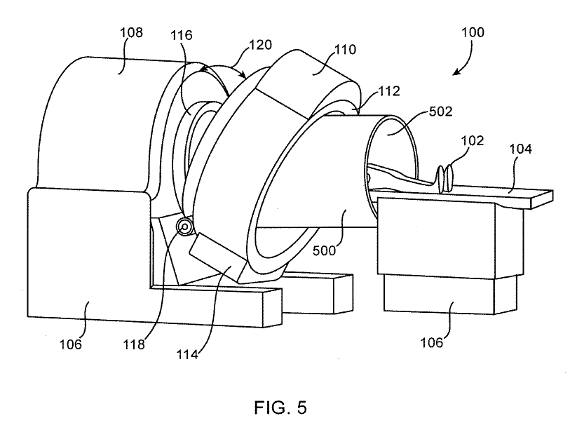

System 100 may further include a protective wall 500 as depicted in FIG. 5. In a preferred embodiment, protective wall 500 is cylindrical in shape, with a patient bore 502. Protective wall 500 may provide further assurance against patient or couch collision with treatment ring 112.

System 100 may be used in conjunction with a treatment planning system that will optimize target treatment with sparing of adjacent critical tissues utilizing the system's fast and versatile coplanar and non-coplanar treatment capabilities. An optimization algorithm may automatically select beam angles and dose intensity from all possible positions from which the system is capable of delivering treatment from treatment head 110. The treatment planning system may also optimize the treatment plan so as to minimize the number of times tilt angle 120 is changed, as such tilting movements are likely to take more time than a rotation of treatment ring 112.

Treatment device 300 may be on, and treating patient 102, during rotation of treatment head 110 or rotating member 116. Alternatively, treatment may be delivered while treatment head 110 and rotating member 116 are stationary, with treatment device 300 turned off during rotation. System 100 may be configured so that rotating member 116 may exceed speeds of one revolution per minute given the design's lack of collision points with patient 102 or patient couch 104, especially when system 100 includes protective wall 500.

While various embodiments in accordance with the disclosed principles have been described above, it should be understood that they have been presented by way of example only, and are not limiting. Thus, the breadth and scope of the invention(s) should not be limited by any of the above-described exemplary embodiments, but should be defined only in accordance with the claims and their equivalents issuing from this disclosure. Furthermore, the above described advantages are not intended to limit the application of any issued claims to processes and structures accomplishing any or all of the advantages.

Additionally, section headings shall not limit or characterize the invention(s) set out in any claims that may issue from this disclosure. Specifically, and by way of example, although the headings refer to a “Technical Field,” such claims should not be limited by the language chosen under this heading to describe the so-called technical field. Further, the description of a technology in the “Background” is not to be construed as an admission that technology is prior art to any invention(s) in this disclosure. Neither is the “Summary” to be considered as a characterization of the invention(s) set forth in issued claims. Furthermore, any reference to this disclosure in general or use of the word “invention” in the singular is not intended to imply any limitation on the scope of the claims set forth below. Multiple inventions may be set forth according to the limitations of the multiple claims issuing from this disclosure, and such claims accordingly define the invention(s), and their equivalents, that are protected thereby.

What is claimed is:

1. A medical treatment system comprising:
a stand;
a rotating member engaged with the stand and configured to rotate in a first rotation plane;
a treatment ring configured to rotate in a second rotation plane, the treatment ring engaged with and angularly displaced from the rotating member by a tilt angle, wherein the first rotation plane and the second rotation plane are angularly separated by the tilt angle;
at least one treatment device engaged with the treatment ring and configured to deliver therapy to a patient; and
wherein rotation of the rotating member causes the at least one treatment device to follow a non-coplanar path for delivering therapy to the patient.

2. The medical treatment system of claim 1 wherein the at least one treatment device is configured to deliver a therapy from the group consisting of radiation therapy, high-frequency focused ultrasound therapy, ablative therapy, laser therapy and radio-frequency therapy.

3. The medical treatment system of claim 1 wherein the tilt angle is variable.

4. The medical treatment system of claim 3 further comprising a beam stopper engaged with the treatment ring and configured to rotate in the second rotation plane, opposite the at least one treatment device.

5. The medical treatment system of claim 1 wherein the tilt angle is fixed.

6. The medical treatment system of claim 5 further comprising a beam stopper engaged with the treatment ring and configured to rotate in the second rotation plane, opposite the at least one treatment device.

7. The medical treatment system of claim 1 wherein a center of the treatment ring defines an isocenter and the at least one treatment device is configured to rotate and deliver therapy to targets displaced from the isocenter.

8. The medical treatment system of claim 1 further comprising at least one imaging device engaged with the treatment ring and located opposite the at least one treatment device.

9. The medical treatment system of claim 1 further comprising at least one imaging device engaged with the treatment ring and located adjacent the at least one treatment device.

10. The medical treatment system of claim 1 further comprising a protective wall around the patient.

11. A medical treatment system comprising:
a stand;
a rotating member engaged with the stand and configured to rotate in a first rotation plane;
a treatment ring engaged with and angularly displaced from the rotating member by a tilt angle and having a first axis;
at least one treatment device to deliver therapy to a patient, engaged with the treatment ring and configured to rotate around the first axis in a second rotation plane,
wherein the first rotation plane and the second rotation plane are angularly separated by the tilt angle; and
wherein rotation of the rotating member causes the at least one treatment device to follow a non-coplanar path for delivering therapy to the patient.

12. The medical treatment system of claim 11 wherein the at least one treatment device is configured to deliver a therapy from the group consisting of radiation therapy, high-frequency focused ultrasound therapy, ablative therapy, laser therapy and radio-frequency therapy.

13. The medical treatment system of claim 11 wherein the tilt angle is variable.

14. The medical treatment system of claim 13 further comprising a beam stopper engaged with the treatment ring and configured to rotate in the second rotation plane, opposite the at least one treatment device.

15. The medical treatment system of claim 11 wherein the tilt angle is fixed.

16. The medical treatment system of claim 15 further comprising a beam stopper engaged with the treatment ring and configured to rotate in the second rotation plane, opposite the at least one treatment device.

17. The medical treatment system of claim 11 wherein a center of the treatment ring defines an isocenter and the at least one treatment device is configured to rotate and deliver therapy to targets displaced from the isocenter.

18. The medical treatment system of claim 11 further comprising at least one imaging device engaged with the treatment ring and located opposite the at least one treatment device.

19. The medical treatment system of claim 11 further comprising at least one imaging device engaged with the treatment ring and located adjacent the at least one treatment device.

20. The medical treatment system of claim 11 further comprising a protective wall around the patient.
